(19) United States
(12) Patent Application Publication
(10) Pub. No.: US 2016/0194455 A1
(43) Pub. Date: Jul. 7, 2016

(54) Silicone Elastomer Gels and Related Hydrosilylation Processes

(71) Applicant: ALZO INTERNATIONAL, INC, Sayreville, NJ (US)

(72) Inventors: Juan R. Mateu, Oak Ridge, NJ (US); Michael R. Batko, Parlin, NJ (US); Nagi Awad, Franklin Lakes, NJ (US); Brianna Bicho, East Brunswick, NJ (US); Michael R. Mosquera, Forked River, NJ (US); Albert A. Zofchak, Holmdel, NJ (US)

(21) Appl. No.: 15/070,673

(22) Filed: Mar. 15, 2016

Related U.S. Application Data
(63) Continuation-in-part of application No. PCT/US14/57365, filed on Sep. 25, 2014.
(60) Provisional application No. 62/137,388, filed on Mar. 24, 2015.
(60) Provisional application No. 61/882,404, filed on Sep. 25, 2013.

(51) Int. Cl. C08G 77/38; A61Q 5/00; A61K 8/895; A61Q 19/00; C08G 77/06; C08G 77/20

(52) U.S. Cl. CPC C08G 77/38; C08G 77/06; C08G 77/20; A61K 8/895; A61Q 19/00; A61Q 5/00; A61K 2800/48

(57) ABSTRACT

The invention provides improved silicone elastomer gels and related hydrosilylation processes and cosmetics. Cross-linked silicone elastomer gels made by processes of the invention exhibit superior characteristics, including improved organic solubility, consistent gelation, stability and enhanced lubricity.

### REFERENCE TO PRIOR APPLICATIONS

This application is a continuation-in-part application which claims the benefit of priority of international application PCT/US2014/57365, filed 25 Sep. 2014 of identical title, which claims priority from U.S. provisional application Ser. No. US61/882,404, filed Sep. 25, 2013 of identical title. This application also claims the benefit of priority of provisional application US/62/137,388, filed 24 Mar. 2015 of identical title. Each of the aforementioned applications is incorporated by reference in its entirety herein.

### FIELD OF THE INVENTION

The invention provides improved silicone elastomer gels and related hydrosilylation processes and cosmetics. Cross-linked silicone elastomer gels made by processes of the invention exhibit superior characteristics, including improved organic solubility, consistent gelation, stability and enhanced lubricity.

### BACKGROUND OF THE INVENTION

Silicone elastomers are well known materials in the cosmetics and personal care products industries. These materials are typically prepared by reacting a polydimethylsiloxane that has several pendant (and often terminal) hydrosilane (e.g., —O—Si(R)(H)—O— as a pendant group or more often —O—Si(R)₂—H as a terminal group) (where R is most desirably methyl) functional groups with vinyl terminated polydimethylsiloxanes to produce a cross-linked three dimensional elastomeric gel structure. As is well known in the industry, the hydrosilane groups readily react with unsaturated groups such as vinyl and allyl groups and other alkenes. Thus, the reaction of a multi-functional hydrosilane polydimethylsiloxane with a dialkene (or multi-alkene) compound will result in a cross-linked polymer. Reactions of this type are detailed in U.S. Pat. No. 6,936,686, which is incorporated in its entirety by reference herein.

The properties of the final elastomer polymer will depend upon the number of hydrosilane and vinyl functional groups contained in each of the starting materials and the molecular weights of each starting polymer. As would be expected, the reaction greatly increases the overall molecular weight of the silicone polymer by forming cross-linking structures between the starting polymers. The cross links also impart elasticity, greatly improve the film forming properties, greatly improve the substantivity and greatly reduce the solubility of the final elastomer in many common solvents. In order to increase the organic solvent compatibility, an alpha olefin (with from 4 to 24 carbons) is often added to the vinyl terminated polydimethylsiloxane reaction component. The alpha olefin can then react with the hydrosilane groups during the elastomeric cross-linking reaction to form pendant alkyl groups. Other olefin containing compounds (such as an ethoxylated allyl alcohol) can be chosen, and added to the vinyl terminated polydimethylsiloxane reaction component. These materials will impart a degree of hydrophilicity making the final silicone elastomer gel more compatible with water and, in some instances, even a water-in-oil (w/o) emulsifier.

For the purpose of making elastomers, the usual practice is to react a bis-divinyl polydimethylsiloxane with a polydimethylsiloxane having multiple pendant methylhydrosilane groups. Thus, the bis-divinyl polydimethylsiloxane becomes the bridging or cross-linking group between the polydimethylsiloxanes that have the hydrosilane groups. Typically, this cross-linking reaction is very fast after addition of the catalyst and, as described in U.S. Pat. No. 6,936,686, visible gelation occurs within about 5 to about 40 minutes after addition of the catalyst. This gelation is a critical point in the production of the polymer (as described in U.S. Pat. No. 6,936,686) after which mixing is stopped to allow the reaction to proceed without disrupting the structure of the gelled matrix.

Further, as is well known in the industry, an organic substrate having multiple unsaturations (such as a polybutadiene or an unsaturated vegetable oil such as soybean or olive oil) can be reacted with multi functional hydrosilane polydimethylsiloxanes to produce an elastomeric gel. This is a well known reaction, but it can be difficult to control reproducibly and, due to compatibility issues between the unsaturated component and the multi functional hydrosilane polydimethylsiloxane, it may not make a clear gel.

Known hydrosilylation processes such as those described in U.S. Pat. No. 6,936,686 are constrained both in terms of unit operation configuration and product characteristics. Known processes are discontinuous: once an elastomeric gel forms as a result of the reaction of a polyorganosiloxane and polyorganohydrosiloxane, mixing is discontinued to facilitate continued reactant cross-linking. A gelling period of several hours without mixing or agitation is often used to allow for a continuation, and hopefully a completion, of the cross-linking process. Discontinuous mixing, however, adversely affects both the cross-linking reaction rate and ultimate product viscosity. In conventional discontinuous mixing processes, mixing cessation leads to an increase in the viscosity of the reaction diluent (a reactant meant to ensure relatively uniform cross-linking throughout the reaction vessel). When the diluent becomes too viscous, it cannot facilitate vessel-wide cross-linking and uniform gelation. To avoid this problem, conventional processes are limited to the use of low-viscosity diluents and yield low viscosity elastomeric products (e.g. elastomers having a viscosity of around 300 centistokes (cst) or less). Known discontinuous processes are limited further by the need to use a diluent to ensure reaction vessel-wide cross-linking.

There is therefore a need for improved hydrosilylation processes that address the aforementioned limitations of known techniques. There is also a need for silicone elastomer gels having a wide viscosity range, Newtonian or non-Newtonian properties, improved stability, as well as other favorable characteristics, including enhanced lubricity.

### OBJECTS OF THE INVENTION

It is an object of this invention to provide improved hydrosilylation processes that enable continuous addition and reaction of unsaturated polyorganosiloxanes and polyorganohydrosiloxanes under mixing conditions that favor substantially uniform reactant cross-linking and the formation of elastomeric gels over a wide viscosity range, Newtonian or non-Newtonian properties, improved stability, as well as other favorable characteristics, including enhanced lubricity.

It is an additional object of this invention to provide improved hydrosilylation processes that enable continuous addition and reaction of unsaturated polyorganosiloxanes and polyorganohydrosiloxanes, thereby reducing processing time and enabling economic one-pot unit operations whose versatility is reflected in one aspect by their optional use of a diluent.

It is a still further object of this invention to provide cross-linked silicone elastomer gels whose viscosity, stability and lubricity impart a wide range of benefits to cosmetic products in which they are used.

Any one or more of these and/or other objects of the invention may be readily gleaned from the description of the invention which follows.

### SUMMARY OF THE INVENTION

We have discovered novel hydrosilylation processes in which a hydrosilylation reaction between unsaturated polyorganosiloxanes and polyorganohydrosiloxanes in a mixer occurs continuously from the gel point to a point of substantial product gelation and yields novel cross-linked silicone elastomer gels having a variety of desirable characteristics, including broad viscosity range, Newtonian or non-Newtonian behavior, improved stability and enhanced lubricity. Processes of the invention reduce processing time, enable economic unit operations and allow for optional use of a diluent. Cross-linked silicone elastomer gels of the invention have a viscosity, stability and lubricity that impart a wide range of benefits to cosmetic products in which they are used.

Compositions according to the present invention exhibit characteristics of increased solubility, consistent gelation characteristics, gel clarity and enhanced stability, as well as increased organic solvent compatibility/solvency as otherwise described herein. Compositions according to the present invention may be modified to provide hydrophobic as well as hydrophilic materials and amines based upon additional chemical components which may be added to the present compositions.

In one embodiment, the invention provides a cross-linked silicone elastomer gel product formed by a hydrosilylation reaction between:
(a) at least one unsaturated polyorganosiloxane;
(b) at least one polyorganohydrosiloxane; and
(c) optionally, at least one vinyl ester and/or alpha olefin;
   wherein the reaction (1) is conducted in the presence of a hydrosilylation catalyst and, optionally, a solvent or diluent (2) is induced upon addition of the at least one unsaturated polyorganosiloxane and the at least one polyorganohydrosiloxane to a mixer at a temperature range of between about 25° C. to about 80° C., more preferably between about 40° C. to about 50° C., even more preferably between about 40° C. to about 45° C. and (3) occurs continuously from the gel point to a point of substantial product gelation.

The term “mixer” as used includes a “mixer-extruder” (e.g. one or more screw mixer extruders e.g. one or more single twin-screw extruders (with multiple extruders arranged in series). Preferably, the mixer-extruder is the “Continuous Processor” (Readco Kurimoto, LLC, York, Pa.). Representative continuous single and twin screw reactor-extruders are also described in U.S. Pat. No. 4,420,603. “Mixer” includes high-shear (rotar/stator) mixers and multi-shaft mixers. The term “mixer” includes two or more mixers of the same or different type that are arranged in series.

“Gel point” is defined in accordance with the Encyclopedia of Polymer Science and Technology (John Wiley & Sons, Inc. 2002) as a liquid-to-solid transition point at which long-range connectivity in the material diverges to infinite size as a result of network formation (“materials at their GP [gel point], called “critical gels” exhibit universal features that merge liquid and solid characteristics into a unique behavior: (1) stress requires an infinite time to relax and (2) relaxation occurs in a broad, self-similar distribution of shorter modes. These features become apparent in rheological experiments that probe the long-time behavior. The broadening of the spectrum has been attributed to two effects, the broadening of the cluster size distribution (molecular clusters, supramolecular clusters) and the branching of the clusters”; id.)

“Substantial product gelation” means the point at which the cross-linked silicone elastomer gel has one or more desired rheological properties (e.g. a viscosity of between about 1,000 to about 4,000,000 or more, about 10,000 to about 3,500,000, about 50,000 to about 3,000,000, about 105,000 to about 4,000,000, about 125,000 to about 3,500,000, about 150,000 to about 3,000,000, about 115,000 to about 2,000,000, about 135,000 to about 1,5000,000 centistokes (cst); a particular gel strength determined, e.g. by calculating the modulus of rigidity); Newtonian behavior (i.e. the gel exhibits constant viscosity irrespective of applied force)).

The hydrosilylation reaction may be conducted in the presence of a solvent or diluent, including, but not limited to the low viscosity silicone oils, hydrocarbon oils, and lower alkanols described in U.S. Pat. No. 6,936,686. Solvents and diluents include, but are not limited to, amphiphilic solvents such as tetrahydrofuran (THF), dioxane, and ethylene glycol dimethylether, hydrocarbon solvents including aliphatic hydrocarbons such as hexane, heptane, cyclohexane, methylcyclohexane, isooctane, and hydrogenated triisobutylene, and aromatic hydrocarbons such as benzene, toluene, xylene, and ethylbenzene, and the like, silicone solvents such as octamethylcyclotetrasiloxane, and decamethylcyclopentasiloxane; and the like.

In certain embodiments, the at least one unsaturated polyorganosiloxane is a bis-divinyl polydimethylsiloxane and the at least one polyorganohydrosiloxane is a polydimethylsiloxane having multiple pendant methylhydrosilane groups. In other embodiments, the divinyl polyorgano siloxane is a divinyl polymethylphenyl siloxane (containing phenyl groups as well as methyl groups) as otherwise described herein and the polyorganohydrosiloxane is a polymethylphenyl siloxane. Compositions made with pendant phenyl groups results in a final silicone elastomer gel which provides a far more shiny coating than when the pendant phenyl groups are excluded from the polymer.

The above-described divinyl polymethylphenyl siloxane can be represented by the formula:

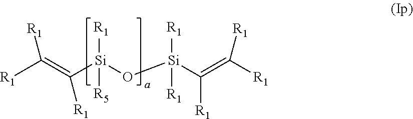

and has a molecular weight of about 20,000 to about 25,000, often about 20,500 to about 24,500, often about 22,000 to about 23,000, often about 22,250 to about 22,750, most preferably about 22,400 to about 22,600) with a being about 265 to about 340 (often about 275 to about 330, more often about 285 to about 320, even more often about 295 to about 305, still more often about 300) and each R₁ being independently H, or an alkyl group of 1 or 3 carbons (preferably methyl) and each R₅ is a phenyl group.

The above-described polymethylphenyl siloxane can be represented by the formula:

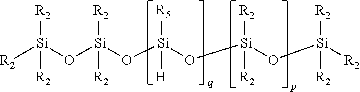

where q is about 5 to about 9; p is about 40 to about 50, each R₂ is independently an alkyl of 1-3 carbon atoms (preferably methyl) and each R₅ is a phenyl group.

A wide variety of hydrosilylation catalysts can be used. Non-limiting examples include the catalysts described in U.S. Pat. Nos. 3,715,334; 3,775,452 (Pt(O) complex with vinyl silicon siloxanes ligands); 3,576,027 (platinum (IV) catalyst prepared by reacting crystalline Platinum (IV) chloropatinic acid and organic silane or siloxanes to form a stable catalyst).

Preferably, the polyorganosiloxane and optional vinyl ester and/or alpha olefin are pre-mixed prior to continuous addition to, and reaction with, the polyorganohydrosiloxane.

In certain embodiments of the invention:
(a) the at least one unsaturated polyorganosiloxane is an α, ω-di lower alkenyl terminated polyorganosiloxane having the formula:

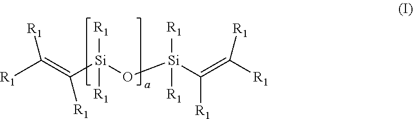

and having a molecular weight of about 20,000 to about 25,000 (preferably about 21,000 to about 24,000, more preferably about 22,000 to about 23,000, even more preferably about 22,250 to about 22,750, most preferably about 22,400 to about 22,600) with a being about 265 to about 340 (preferably about 275 to about 330, more preferably about 285 to about 320, even more preferably about 295 to about 305, still more preferably about 300) and each R₁ being independently H, or an alkyl group of 1 or 3 carbons; and
(b) the at least one polyorganohydrosiloxane has the formula:

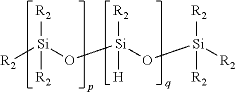

and a molecular weight of about 3,500 to 4,000 (preferably about 3,600 to about 3,900, more preferably about 3,700 to about 3,800, still more preferably about 3,725 to about 3,775, still more preferably about 3,740 to about 3,760), where q is about 5 to about 9; p is about 40 to about 50, and each R₂ is independently an alkyl of 1-3 carbon atoms. As noted and described above, each of the α, ω-di lower alkenyl terminated polyorganosiloxane and polyorganohydrosiloxane can contain phenyl groups substituted for certain of the methyl groups in order to provide polymeric materials which provide coatings exhibiting substantially greater shine than coatings which do not contain phenyl substituent groups.

In still other embodiments, the invention provides cross-linked silicone elastomer gels formed by a hydrosilylation reaction between:
(a) at least one alpha-olefin of the formula (CH₂)═R³R⁴, where R³ is H or an alkyl group containing 1-40 carbon atoms, and R⁴ is an alkyl group containing 1-40 carbon atoms; and/or an alpha, omega-diene of the formula (CH₂)═CH(CH₂)ₓCH═CH₂, where x is 1-20; and (b) at least one polydimethylsiloxane having multiple pendant methylhydrosilane groups;
   wherein the reaction (1) is conducted in the presence of a hydrosilylation catalyst and, optionally, a solvent or diluent, (2) is conducted between (a) and (b) induced upon addition of the at least one alpha-olefin and the at least one polydimethyl/methylsiloxanedimethylsiloxane to a mixer at a temperature range of between about 25° C. to about 80° C., more preferably between about 40° C. to about 50° C., even more preferably between about 40° C. to about 45° C., and (3) occurs continuously from the gel point to a point of substantial product gelation.

In still other embodiments, the invention provides cross-linked silicone elastomer gels formed by a hydrosilylation reaction between:
(a) at least one multi-vinyl functional hydrocarbon;
(b) at least one bis-dihydrosilane polydialkylsiloxane; and
(c) optionally, an allyl alcohol ethoxylate;
   wherein the reaction (1) is conducted in the presence of a hydrosilylation catalyst and, optionally, a solvent or diluent (2) is induced upon addition of the at least one multi-vinyl functional hydrocarbon, the at least one bis-dihydrosilane polydialkylsiloxane and, optionally, the allyl alcohol ethoxylate to a mixer at a temperature range of between about 25° C. to about 80° C., more preferably between about 40° C. to about 50° C., even more preferably between about 40° C. to about 45° C. and (3) occurs continuously from the gel point to a point of substantial product gelation.

In certain embodiments, the multi-vinyl functional hydrocarbon has the formula:

HC₃—HC═CH—CH₂—(CH₂—HC═CH—CH₂)ⱼCH₂—HC═CH—CH₃

where j is an integer from 5 to 500.

In still other embodiments, the multi-vinyl functional hydrocarbon is a polybutadiene comprising at least about 90% by weight of cis olefins.

In certain embodiments, the bis-dihydrosilane polydialkylsiloxane has the formula:

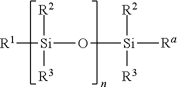

where R¹ and R^{a} are each H; each R² and R³ is independently a C₁-C₁₀ alkyl group; and n is from 5 to 50,000.

A cross-linked silicone elastomer gel of the invention can further comprise about 0.01% to about 7.5% by weight of ally! alcohol ethoxylate units (e.g. allyl alcohol ethoxylate units comprising about 5 to about 100 ethylene glycol units), and may also further comprise about 0.01% to about 7.5% by weight of a polyurethane.

Notably, as the cross-linking reactions yield a silicone elastomer gel whose viscosity is approaching non-Newtonian levels, a continuous processor (mixer) used in certain embodiments of the invention can act as an extruder that kneads or mixes the silicone elastomer until it is extruded into a holding vessel. In contrast, when such conditions are encountered in conventional processes, all mixing or agitation is stopped and the reaction proceeds for several hours in the vessel without mixing or agitation. Continuous processing in accordance with the invention allows for mixing after the gelation point of the reaction. Once the gel is extruded out of the continuous processor (mixer), the gel is ready for further dilution, e.g. by using a homogenizer.

These and other aspects of the invention are described further in the Detailed Description of the Invention.

### DETAILED DESCRIPTION OF THE INVENTION

In addition to the definitions provided above, the following definitions also apply.

The term “patient or subject” is used to describe a mammal, including a human to which compositions according to the present invention may be applied.

The term “effective” is used, in context, to describe an amount or concentration of a compound, composition or component, as otherwise described herein which is included or used to provide an intended effect or trait, such as emulsification (emulsifiers), emolliency, wettability, skin adherence, storage stability, viscosity and/or solubility to a formulation of a personal care product or are used to produce a compound or composition according to the present invention.

The term “personal care product” or “personal care composition” is used to describe a chemical composition used for the purpose of cleansing, conditioning, grooming, beautifying, or otherwise enhancing the appearance of the human body, especially keratinous tissue, including skin, nails and hair. Personal care products include skin care products, cosmetic products, antiperspirants, deodorants, toiletries, perfumes, soaps, bath oils, feminine care products, hair-care products, oral hygiene products, depilatories, including shampoos, conditioners, hair straightening products and other hair care products, color cosmetics such as lipsticks, creams, make-ups, skin creams, lotions (preferably comprised of water-in-oil or oil-in-water emulsions), shave creams and gels, after-shave lotions and shave-conditioning compositions and sunscreen products, among numerous others.

Personal care products according to the present invention comprise an admixture of a silicone cross linked hydrocarbon elastomer as otherwise described herein alone or optionally in combination with an oil and water (to produce an emulsion which may be further added to other components to produce a personal care composition) in the weight percentages as otherwise disclosed herein and at least one or more additional component selected from the group consisting of an aqueous solvent (e.g. alcohol or other compatible solvent), a non-aqueous solvent, emollients, humectants, oils (polar and non-polar), conditioning agents, surfactants, thickeners/thickening agents, stiffening agents, emulsifiers, including secondary emulsifiers, medicaments, fragrances, preservatives, deodorant components, anti-perspirant compounds, skin protecting agents, pigments, dyes, coloring agents, sunscreens and mixtures thereof, among others.

Preferred personal care products according to the present invention comprise about 0.01% to about 95% by weight of an emulsion which comprises a silicone cross-linked hydrocarbon elastomer as otherwise described herein, an oil and water, with the remainder of the composition comprising at least one additional component selected from the group consisting of an aqueous solvent (e.g. alcohol or other water compatible solvent), a non-aqueous solvent, emollients, humectants, a secondary oils (polar and non-polar), conditioning agents, emulsifiers, including secondary emulsifiers, surfactants, thickeners, stiffening agents, medicaments, fragrances, preservatives, deodorant components, anti-perspirant compounds, skin protecting agents, pigments, sunscreens and mixtures thereof, among others.

The term “silicone cross linked hydrocarbon elastomer” or “silicone cross-linked hydrocarbon polymer” describes a multi alkene functional compound which may or may not be a polymer (preferably, polybutadiene or a multi-unsaturated polyurethane, more preferably polybutadiene) which is cross linked (or chain-extended) with a bis-hydrosilane terminated polysiloxane and exhibits favorable characteristics of gelation, solubility and stability. The polyorganosiloxane polymer (bis-hydrosilane silicone polymer) which is cross linking (or chain-extending) the hydrocarbon according to the present invention may vary significantly in chemical composition but preferably is a polymeric composition comprised of

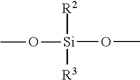

units, where R² and R³ are each independently a C₁-C₁₀ alkyl (preferably C₁-C₃ alkyl, more preferably methyl) (as described below), and optionally, in a small number of instances in certain embodiments as otherwise described herein, Si—H groups or hydroxyl groups, and may vary in average molecular weight M_{w} from about 1,000 to about 1,500,000 or more, preferably about 1,000 to about 100,000, more preferably about 2,500 to about 25,000 or more, depending upon the final viscosity and other characteristics desired.

Silicone cross linking agents (bis-hydrosilane terminated polyalkylsiloxanes) described herein may comprise as little as 2% and as much as 98% by weight of the final silicone cross-linked hydrocarbon elastomer, the remainder comprising the multifunctional hydrocarbon compound, but in preferred aspects the silicone cross-linking agent comprises about 50% to about 98% of the final silicone cross-linked hydrocarbon elastomer and the multifunctional hydrocarbon compound (e.g. polybutadiene) comprising about 0.1% to about 25%, about 0.25% to about 20%, about 0.5% to about 15%, about 1% to about 10% by weight of the final silicone cross-linked hydrocarbon elastomer. In preferred aspects, the multifunctional hydrocarbon comprises about 0.1% to about 10% by weight of the silicone cross-linking agent used in the preparation of the silicone cross-linked hydrocarbon elastomers of the present invention. As further described in greater detail herein, the silicone cross-linked hydrocarbon elastomers may also comprise allyl alcohol ethoxylate units and/or polyurethane units.

Silicone polymers according to the present invention which are used to produce silicone cross-linked hydrocarbon elastomers preferably comprise one Si—H group at each of the distil ends of the elastomer (e.g. bis-hydrosilane polydimethylsiloxane) which are capable of cross-linking with multi vinyl functional hydrocarbon as otherwise described herein (e.g. polybutadiene, unsaturated polyurethane, among others).

In preferred aspects, an allyl alcohol ethoxylate may optionally comprise (in the final polymer) an amount of about 0.01% to about 7.5%, about 0.05% to about 5%, about 0.1% to about 1% by weight of the monomers/polymers which ultimately form certain embodiments of the silicone cross-linked hydrocarbon elastomer according to the present invention. The inclusion of allyl alcohol ethoxylate may increase the hydrophilicity of the final silicone cross-linked hydrocarbon elastomers according to the present invention. In certain other aspects, a polyurethane polymer also may be added to the multifunctional unsaturated hydrocarbon crosslinkable agent and reacted with the bis-hydrosilane polyorganosiloxane polymer to provide final hydrophilic silicone cross-linked hydrocarbon elastomers. The polyurethane polymer comprising (when optionally present) about 0.01% to about 15%, about 0.05% to about 10%, about 0.05% to about 5% or more by weight of the final polymeric composition in order to provide a further hydrophilic/skin adhering component, solubilizer or UV absorbing component.

The final silicone cross-linked hydrocarbon polymers (which may optionally include allyl alcohol ethoxylate and/or polyurethane units to increase hydrophilicity or, in the case of polyurethanes, hydrophilic, skin-adherent, solubilizing or UV absorbing qualities of the final polymers) according to the present invention are multifunctional unsaturated hydrocarbon compounds, including polybutadiene which are cross-linked with a bis-hydrosilane terminated polysiloxane compound (the reaction preferably occurring between the olefinic groups on the multiply unsaturated hydrocarbon and the Si—H groups and, in some cases, optional alkenyl groups on the cross-linking silicone polymer. Optionally, the cross-linking bis-hydrosilane polydimethylsiloxane may be reacted with an unsaturated polymeric silicone compound, an alpha olefin and/or an allyl alcohol ethoxylate prior to cross-linking with the multiply unsaturated hydrocarbon compound. For example, polydimethylsiloxanes with several pendant hydrosilane groups may be used to introduce an allyl alcohol ethoxylate (each allyl alcohol monomer preferably containing from 5 to about 100, about 10 to about 50, about 15 to about 45, about 10 to about 65, about 15 to about 25, about 50 to about 100, about 65 to about 85, about 75 ethoxylate/ethylene glycol units) monomer into the final silicone cross-linked hydrocarbon polymer. These groups can also be used to introduce polyurethane or polyester compounds having the appropriate unsaturated group. Alternatively, the hydrophilic silicone elastomer (hydrophilic through introduction of allyl alcohol ethoxylate groups) and/or polyurethane or polyester may simply be admixed without further cross-linking/polymerization.

In certain preferred aspects of the present invention, the final silicone cross-linked hydrocarbon polymer is prepared from a reaction mixture which comprises a hydrosilane terminated polydimethylsiloxane polymer as described above (which may optionally further comprise an allyl alcohol ethoxylate group as described herein and/or a reactive polyurethane or polyester wherein the hydrosilane terminated polydimethylsiloxane and the allyl alcohol ethoxylate and/or polyurethane or polyester are covalently linked) as a cross-linking agent. This cross-linking agent is then reacted with a multifunctional unsaturated hydrocarbon such as polybutadiene as described herein. The polybutadiene itself may be optionally mixed or combined with an allyl alcohol ethoxylate and/or a polyurethane or polyester prior to reaction with the hydrosilane terminated polydimethylsiloxane cross-linking agent to form the final silicone cross-linked hydrocarbon polymer according to the present invention. Thus, silicone cross-linked hydrocarbon polymers according to the invention comprise the reaction product of a cross-linking silicone polymer as otherwise described hereinabove that contains hydrosilane groups at the distil ends of the polysiloxane, as well as an optional allyl alcohol ethoxylate component and/or an optional polyurethane or polyester component. Each of the optional allyl alcohol ethoxylate component and the optional polyurethane or polyester component independently comprise about 0.1% to about 75%, about 0.5% to about 50%, often about 0.5% to about 10% or 1% to about 7.5% by weight of the bis-hydrosilane polydimethylsiloxane cross-linking agent which is reacted with the multifunctional hydrocarbon polymer backbone.

Alternatively, the final silicone cross-linked hydrocarbon elastomers comprise the reaction product of a cross-linking silicone polymer as otherwise described herein (i.e., without allyl alcohol ethoxylate and/or a polyurethane) with a multiply unsaturated hydrocarbon (e.g., polybutadiene) which may optionally include an allyl alcohol ethoxylate and/or a polyurethane as described above (preferably comprising about 0.01% to about 7.5%, about 0.05% to about 5%, about 0.1% to about 1% by weight of the multifunctional hydrocarbon.

For preparation of silicone cross-linked hydrocarbon elastomers which contain a bonded polyurethane to optionally instill at least a portion of hydrophilic, self-adhering, solubilizing and/or UV absorbing character to the final silicone elastomer, the polyurethane compound comprises about 0.01% to about 7.5%, about 0.01% to about 5%, about 0.05% to about 1% of the final hydrocarbon silicone cross-linked hydrocarbon elastomer.

In certain preferred embodiments, the bis-hydrosilane polydimethylsiloxanes (silicone polymer crosslinkers) which are used to prepare silicone cross-linked hydrocarbon elastomers according to the present invention have the following structure:

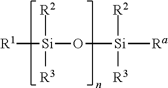

Where R¹ and R^{a} are each independently H groups;

Each R² and R³ is independently a C₁-C₁₀ alkyl group (preferably C₁-C₃ alkyl, preferably methyl); and

n is from 5 to 50,000, about 10 to about 25,000, about 100 to about 10,000, about 100 to about 5,000, about 200 to about 5,000, about 500 to about 2,500.

Silicone cross-linked hydrocarbon elastomers are generally formed by reacting a polysiloxane polymer which contains two Si—H bonds at distil ends of the molecule (a his hydrosilane polydialkylsiloxane as otherwise described herein) with a multifunctional hydrocarbon (e.g. polybutadiene), each of which is reactive with a Si—H group. The multifunctional hydrocarbon may vary in size, but generally ranges in size from a molecular weight of several hundred to 25,000 or more, with a preferred molecular weight range of at least about 500 to about 10,000, about 1,500-7,500, about 2,000-5,000 or about 2,500.

The term “cross-linking” is used to describe the reaction of the silicone polymer with the multifunctional hydrocarbon backbone in the present compositions. It is noted that the silicone polymer often has only two functional groups, e.g. a Si—H group on each of the distil ends of the silicone polymer, the polymer may also be referred to as a chain extender or chain extending agent. However, it will be understood the term cross-linking may be used to refer to the silicone polymer or crosslinker used in the present invention and the reaction of the silicone polymer or crosslinker with the (multiply unsaturated) hydrocarbon.

The term “polybutadiene” shall mean, within the context of its use, a polymeric material which is produced from butadiene monomers. Polybutadiene polymers for use in the present invention have a structure according to the chemical formula:

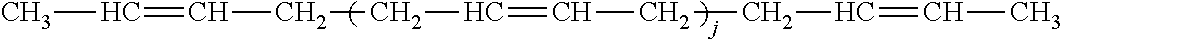

Where j is from 5 to about 500 or more, about 16 to about 200; about 30 to about 150, about 40 to about 100, about 90-110, about 100. Preferred polybutadiene polymers for use as multifunctional hydrocarbon polymer backbone herein comprise about 5% to about 50% by weight of olefinic character (also referred to as “vinyl content”-based upon the molecular weight of olefin within the polybutadiene molecule), about 5 to about 35% by weight olefin, about 15% to about 25% by weight olefin. Preferred polybutadiene polymers for use in the present invention comprise about 90+% cis olefins (of a mixture of cis and trans olefins within the polybutadiene molecule), about 95+% cis olefins, about 99+% cis olefins, about 99.5+% cis olefins, about 99.9+% cis olefins. It is noted that the polybutadiene component of the present invention contains a number of vinyl groups which may react with Si—H or other groups (as otherwise described herein) within the silicone elastomer cross-linking agents to produce silicone cross-linked hydrocarbon elastomers according to the present invention. In the present invention, it is contemplated that the multifunctional hydrocarbon (polybutadiene), especially including polybutadiene functions as a hydrocarbon backbone in the silicone cross-linked hydrocarbon elastomer polymers according to the present invention.

Preferred polybutadiene polymers for use in the present invention comprise about 0.005% to about 7.5% by weight of the final silicone cross-linked hydrocarbon elastomer, about 0.05% to about 5% by weight, about 0.1% to about 2.5% by weight, about 0.25 to about 4%.

Because of the physicochemical characteristics of polybutadiene and its ability to react with hydrosilane terminated polydimethylsiloxanes, the compatibility of polybutadiene as a hydrocarbon backbone with silicone cross-linking agents/chain extenders as otherwise described herein is exceptional and results in final compositions which can be manufactured with a high degree of purity, consistency, gelation characteristics, flexibility and compatibility for inclusion in personal care products. It is noted that the inherent high compatibility between the polybutadiene polymer backbone and the silicone crosslinkers/chain extenders (of varying compositions as otherwise described herein) provides an easily and consistently manufactured silicone cross-linked hydrocarbon elastomer which can be varied quite markedly in final characteristics by incorporation of additional components (such as allyl alcohol ethoxylate and polyurethanes) as otherwise described herein.

The term “polyurethane” shall mean, within the context of its use, a polymeric urethane compound comprising at least one and preferably, two or more urethane linkages which are generally formed by reacting at least one compound containing a free alcohol (primary, secondary or tertiary), preferably at least one compound containing at least two alcohol groups (“polyol”) and a diisocyanate compound. Thus, the term polyurethane as used herein incorporates dimer urethanes (those compounds which contain two urethane bonds) which are formed from a diisocyanate and a monohydric alcohol of varying structure, which structure may contain, for example, an active group or a protected active group such as a silyl-protected hydroxyl group or amine group wherein the protecting group may be removed subsequent to formation of the polyurethane or an olefinic group (such as for example, a vinyl group, acrylate or methacrylate group) which can participate in a reaction with a silane group from the silicone polymer crosslinker to produce a silicone cross-linked hydrocarbon elastomer/polyurethane composition.

In addition, polyurethanes according to the present invention preferably are formed by reacting at least one polyol (a compound which is either hydrocarbon or siloxane based and which contains at least two free hydroxy groups with a diisocyanate to produce a polyurethane, with the polyol optionally and preferably containing at least one functional group which does not participate in the polymerization reaction to form the polyurethane composition, but which, subsequent to the polymerization reaction, can be used to crosslink the polyurethane composition to a silicone elastomer in preferred compositions according to the present invention. In preferred aspects of the invention, polyurethane compounds which are reacted with a silicone elastomer to produce hydrophilic silicone elastomers preferably have sufficient hydrophilic character (for example, by containing sufficient hydroxyl groups and/or ethoxylated-polyethylene oxide or PEG groups) to instill hydrophilic character to the final hydrophilic silicone elastomers according to the present invention.

Preferred urethane polymers according to the present invention have the general structure V:

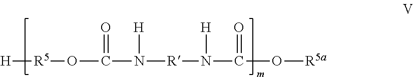

Where R⁵ is an optionally substituted hydrocarbon or optionally substituted siloxane group, preferably, an optionally substituted (with hydroxyl groups and/or PEG groups comprising from 1 to 100 or 2 to 25 ethylene oxide units) C₁-C₅₀ hydrocarbon group containing at least one olefinic group or a polyethylene oxide group comprising between 1 and 500, 2 and 100, 5 and 25, 5 and 20, 5 and 15 ethylene oxide groups which may be optionally endcapped with or contain a polymerizable group such as an alkenyl or (meth)acrylate group, or a siloxane group according to the structure:

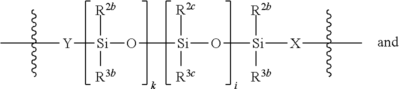

R^{5a} is an optionally substituted hydrocarbon (which may contain hydroxyl and/or PEG groups as otherwise described here) or a siloxane group, preferably, an optionally substituted C₁-C₅₀ hydrocarbon group, optionally containing at least one olefinic group, or a siloxane group according to the structure:

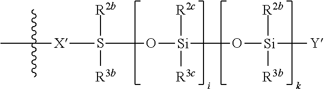

Wherein Y is absent, O or a —W—(OZ)ᵣ-Q-(CH₂)_{q}-T- group;
X is absent or a -T-(CH₂)_{q}—Q—(ZO)ᵣ—W′— group;
X′ is absent or a —W′—(OZ)ᵣ-Q-(CH₂)_{q}-T- group;
Y′ is absent or a -T-(CH₂)_{q}-Q-(ZO)ᵣ—W²— group;
W is absent when r is an integer of 1 or more and W is absent or 0 when r is 0;
Q is absent or 0;
q is an integer from 0 to 10, preferably 1 to 6, preferably 1 to 3;
r is an integer from 0 to 100, 0 to 40, preferably 1 to 20 or 1 to 10, with the proviso that q or r is at least 1;
T is absent or O;
W′ is absent when r is 0 and is a Z group when r is 1 or more;
W² is H;
Z is independently an ethylene group, a propylene group or a mixture of ethylene and propylene groups;
R^{2b} and R^{3b} are each independently a C₁-C₁₀ alkyl group (preferably both are a C₁-C₃ alkyl group, preferably both are methyl groups), preferably R^{2b} and R^{3b} are both C₁-C₁₀ alkyl groups, preferably C₁-C₃ alkyl groups, preferably both are the same C₁-C₃ alkyl group, preferably both are methyl groups;
R^{2c} and R^{3c} are independently selected from an optionally substituted C₁-C₆ alkyl group (substitution with OH or a C₁-C₃ alkyl group which itself may be optionally substituted with a hydroxyl group) and optionally, an Si—H group, an alkenyl group and/or a hydroxyl group in small percentages of the total number of R^{2c} and R^{3c} substituents within the polymer. In certain embodiments, R^{2b}, R^{3b}R^{2c} and R^{3c} optionally may comprise a small percentage (i.e., less than about 2%, 1.5%, 1.0%, 0.75%, 0.5%, 0.25%, 0.1%, 0.05% or 0.002%) of Si—H groups, alkenyl groups and/or hydroxyl groups of the total number of R^{2b}, R^{3b}R^{2c} and R^{3c} groups which are found in the silicone group;
R′ is an optionally substituted C₂ through C₃₆ (preferably, C₆ through C₂₂, most preferably an isophorone group) linear, cyclic or branch-chained saturated or unsaturated hydrocarbon group (which may be monomeric or dimeric, an aromatic group, including a phenyl or benzyl group or substituted phenyl or benzyl group, an alkylphenyl, alkylbenzyl or substituted alkylphenyl or alkylbenzyl group);
i is an integer from 0 to 50, preferably 0, 1, 2, 3, 4, 5, 6, 7, 8, 9 or 10 (preferably 0 or 1);
k is an integer from 0 to 100, 1 to 100, about 5 to 50, about 10 to 45, preferably about 20 to 40; and
m is from 1 to 100,000, about 1 to 25,000, about 5 to 25,000, about 50 to 20,000, about 50 to 20,000, about 100 to 20,000, about 100 to 10,000, about 200 to 5,000, about 250 to 2,500, about 500 to about 2,000, 1 to about 1,000, 1 to about 750, 2 to about 650, about 50 to 15,000, about 10 to 10,000, about 200 to 5,000, about 250 to about 2,500, about 5 to about 150, about 3 to 100, about 5 to 250. Preferably, the polyurethane according to the present invention is obtained by reacting a polyol (which may be hydrocarbon based or siloxane based and contains at least two hydroxyl groups) with a diisocyanate compound to produce a polyurethane composition accordingly.

In certain preferred aspects of the present invention in the polyurethane formula V described above, R⁵ is a O—R⁶ group and R^{5a} is a R^{6a}—OH group where R⁶ and R^{6a} are each independently an optionally substituted hydrocarbon or an optionally substituted siloxane group as set forth for R⁵ and R^{5a}, respectively and generally described above.

One or more polyols and/or diisocyanates may be used to produce polyurethane polymers according to the present invention, with preferred polyols having, in addition to having at least two free hydroxy groups to participate in polymerization reactions to form polyurethanes, at least one reactive alkene (unsaturated hydrocarbon) group must be available for reaction with the hydrosilane terminated polydimethylsiloxane cross-linking agents of the present invention, and with the diisocyanate preferably being isophorone diisocyanate. Further preferred polyols contain multiple hydroxyl groups or alternatively, polyethylene oxide groups wherein the PEG groups contain from 2 to 100 ethylene oxide groups, preferably 3 to 50, 5 to 25 or 5 to 10.

Alternative polyurethanes according to the present invention also are prepared from a diisocyanate, preferably isophorone diisocyanate, glycerin and glycerin esters, propylene glycol and its esters, dipropylene glycol and its esters, alkyl amines, ethoxylated alkyl amines, propoxylated alkyl amines, silicone ethoxylates and silicone propoxylates, among others.

The term “polyol” refers to a hydrocarbon or siloxane based compound having at least two free hydroxyl groups which can participate in a reaction with diisocyanate to provide a polyurethane composition. In preferred aspects of the invention, a polyol according to the present invention, in addition to the two free hydroxyl groups which react with diisocyanate compounds, also contains an additional “reactive functional group” which, subsequent to the formation of the polyurethane compound, may participate in a cross-linking reaction with a reactive functional group on a silicone polymer admixed with the polyurethane, to produce silicone cross-linked hydrocarbon/polyurethane elastomer compositions.

The term “monohydric alcohol” refers to a compound containing a single hydroxyl group which may react with a diisocyanate compound to produce dimer urethane compounds according to the present invention. Monohydric alcohols advantageously contain at least one reactive functional group which, after formation of the dimer urethane, can react with a reactive group on a silicone polymer admixed with the dimer urethane to produce a silicone cross-linked hydrocarbon/polyurethane elastomer compositions.

The polyol(s) used to polymerize with diisocyanate may vary widely in character from hydrophilic (polar) to hydrophobic (non-polar), but are preferably hydrophilic in nature. Although a large number of polyols can be used to produce polyurethane compositions according to the present invention, preferred polyols include triglycerides which contain fatty acids having free hydroxyl groups and/or olefinic groups such as castor oil triglycerides or other triglycerides, glycerol, substituted glycerols or polyglycerols such as C₁₀-C₂₄ di-fatty polyglycerol (preferably, polyglycerol-2-diisostearate), di-fatty alkanolmonoglycerol, such as glycerol diricinoleate, polyethylene glycol alkylamines, especially polyethyleneglycol fatty amines, such as PEG-15 cocamine, or di-PEG-15 soyamine or related dipolyethylene glycol fatty amines, including di-PEG soyamine, polyethyleneglycol (PEG), substituted polyethyleneglycol, polydialkylsiloxane such as polydimethylsiloxane (e.g. dimethicone), or a di-polyethyleneglycol dimethicone, or related polysiloxane and bis-hydroxy terminated polybutadienes. Polyols are polymerized with a diisocyanate compound, preferably isophorone diisocyanate.

In an alternative embodiment, multiply unsaturated polyols, such as hydroxyl-terminated polybutadiene may be reacted with a diisocyanate to form a multiply unsaturated hydrocarbon/polyurethane that can then be cross-linked with α,ω-hydrosilanepolydimethyl siloxane to form a silicone cross-linked polyurethane elastomer. Further, the hydroxyl terminated polybutadiene may be reacted with an acid, acid anhydride or acid halide to form a diester that can then be cross-linked with α,ω-hydrosilanepolydimethyl siloxane to form a silicone cross-linked hydrocarbon/ester elastomer. Alternatively, the hydroxyl terminated polybutadiene may be reacted with ethylene oxide, propylene oxide and the like to form a polyether that can then be cross-linked with α,ω-hydrosilanepolydimethyl siloxane to form a silicone cross-linked hydrocarbon/polyether elastomer.

The term “polyester” is used throughout the specification to describe a polymer which may be incorporated into compositions herein, in addition to a polyurethane as otherwise ssdescribed herein or as an alternative to a polyurethane. Polyesters may be formed by reacting monomeric compounds which are diols (of a wide variety including silicone containing diols) with diacids (varying widely, but often an organic acid having between 2 and 20 carbon atoms) or alternatively one or more monomers which contain a hydroxyl group and an acid, such that an ester group may be formed by the reaction of a hydroxyl group with an acid, thus forming a polyester. Polyesters which may be used according to the present invention may vary widely depending upon the physicochemical characteristics which are to be included into compositions according to the present invention. Polyesters may be incorporated into compositions according to the present invention at a free hydroxyl group or free carboxyl acid group which may be used to start a polymerization reaction to produce a polyster sidechain. Alternative approaches are also provided and include, for example, reacting an allyl alcohol moiety with one or more of the functional groups on the cross-linked silicone polymer and then forming a polyester off of the free alcohol group from the reacted allyl alcohol.

The term “diisocyanate” is used throughout the specification to describe a linear, cyclic or branch-chained hydrocarbon having two free isocyanate groups. The term “diisocyanate” also includes halogen substituted linear, cyclic or branch-chained shydrocarbons having two free isocyanate groups. Exemplary diisocyanates include, for example, isophoronediisocyanate, m-phenylene-diisocyanate, p-phenylenediisocyanate, 4,4-butyl-m-phenylene-diisocyanate, 4-methoxy-m-phenylenediisocyanate, 4-phenoxy-m-phenylenediisocyanate, 4-chloro-m-phenyldiisocyanate, toluene diisocyanate, m-xylylene diisocyanate, p-xylylene diisocyanate, 1,4-napthalene diisocyanate, cumene-1,4-diisocyanate, durene diisocyanate, 1,5-napthylene diisocyanate, 1,8-napthylene diisocyanate, 1,5-tetrahydronapthylene diisocyanate, 2,6-napthylene diisocyanate, 1,5-tetrahydronapthylene diisocyanate; p,p-diphylene diisocyanate; 2,4-diphenylhexane-1,6-diisocyanate; methylene diisocyanate; ethylene diisocyanate; trimethylene diisocyanate, tetramethylene diisocyanate, pentamethylene diisocyanate, hexamethylene diisocyanate, nonamethylene diisocyanate, decamethylene diisocyanate, 3-chloro-trimethylene diisocyanate and 2,3-dimethyltetramethylene diisocyanate, among numerous others. Isophorone diisocyanate is the preferred diisocyanate used in the present invention.

The term un-substituted when used in context is used to describe a hydrocarbon moiety such as an alkyl group or alkene or other group which contains only hydrogen atoms bonded to carbons within the moiety. It can include aryl (aromatic groups such as phenyl) groups, as well. The term un-substituted is used in context to describe a hydrocarbon moiety which is substituted, i.e., it contains, within the context of its use, a pendant hydroxyl group (in preferred aspects numerous alcohol groups, an ether group (such as within a glycol or polyglycol/(PEG), glycerol or polyglycerol or other group), a keto group, an amine (which may itself be substituted with alkyl groups, including fatty (C₈-C₃₀) alkyl groups or alkanol groups, for example), an alkyl or alkene group attached to a carbon atom of the moiety. The number of carbon atoms within a substituent group may vary from 0 to 30 or more, 0 to 24 or more, 0 to 18, 0 to 12, 0 to 10, 1 to 8, and 1 to 6 and may contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more carbon atoms, depending upon the context of the use of the compound to which the substituent is attached. The term “optionally” means that a particular component, substituent or the like may or may not be present, depending upon the context within which the component, substituent or the like is used.

The term “oil” is used throughout the specification to describe any of various lubricious, hydrophobic and combustible substances obtained from animal, vegetable and mineral matter. These are used in combination with silicone cross-linked hydrocarbon elastomers to provide emollient characteristics (with from 1% by weight to 99% by weight of a combination of hydrocarbon elastomer and oil). Alternatively, the hydrocarbon elastomers may be used to form emulsions of the present invention by combining an effective amount of an oil and water with the silicone cross-linked hydrocarbon elastomers according to the present invention to provide oil-in-water or water-in-oil emulsions which may be used alone or in lotions or creams in certain aspects of the invention. The amount of hydrocarbon elastomer in emulsions will range from about 0.1% to about 50%, about 0.25% to about 25%, about 0.5% to about 20%, about 1% to abut 15%, about 2% to about 10%, about 0.75% to about 5% by weight; the amount of oil will range from about 0.1% to about 50%, about 0.25% to about 25%, about 0.5% to about 20%, about 1% to about 15%, about 2% to about 10%, about 0.75% to about 5% by weight and the amount of water will range from about 0.25% to about 95%, about 0.5% to about 85%, about 0.75% to about 80%, about 1% to about 75%, about 2% to about 70%, about 5% to about 65%, about 1% to about 15%, about 2% to about 10%, about 0.75% to about 5% by weight; about 45% to about 99% by weight of the emulsion produced.

Emollient oils for use in the present invention may include petroleum-based oil derivatives such as purified petrolatum and mineral oil. Petroleum-derived oils include aliphatic or wax-based oils, aromatic or asphalt-based oils and mixed base oils and may include relatively polar and non-polar oils. Non-polar oils are generally oils such as petrolatum or mineral oil or its derivatives which are hydrocarbons and are more hydrophobic and lipophilic compared to synthetic oils, such as esters, which may be referred to as polar oils. It is understood that within the class of oils, the use of the terms non-polar and polar are relative within this very hydrophobic and lipophilic class, and all of the oils tend to be much more hydrophobic and lipophilic than the water phase which is used to produce the water-in-oil emulsion of the present invention. Preferred hydrophobic oils for use in the present invention include mineral oil and petrolatum. Preferred less hydrophobic (i.e., more polar) oils for use in the present invention include a number of maleates, neopentanoates, neopentanoyls, citrates and fumarates, and any other cosmetically acceptable ester emollient.

In addition to the above-described oils, certain essential oils derived from plants such as volatile liquids derived from flowers, stems and leaves and other parts of the plant which may include terpenoids and other natural products including triglycerides may also be considered oils for purposes of the present invention.

Petrolatum (mineral fat, petroleum jelly or mineral jelly) and mineral oil products for use in the present invention may be obtained from a variety of suppliers. These products may range widely in viscosity and other physical and chemical characteristics such as molecular weight and purity. Preferred petrolatum and mineral oil for use in the present invention are those which exhibit significant utility in cosmetic and pharmaceutical products. Cosmetic grade oils are preferred oils for use in the present invention.

Additional oils for use in the present invention may include, for example, mono-, di- and tri-glycerides which may be natural or synthetic (derived from esterification of glycerol and at least one organic acid, saturated or unsaturated, such as for example, such as butyric, caproic, palmitic, stearic, oleic, linoleic or linolenic acids, among numerous others, preferably a fatty organic acid, comprising between 8 and 26 carbon atoms). Glyceride esters for use in the present invention include vegetable oils derived chiefly from seeds or nuts and include drying oils, for example, linseed, iticica and tung, among others; semi-drying oils, for example, soybean, sunflower, safflower and cottonseed oil; non-drying oils, for example castor and coconut oil; and other oils, such as those used in soap, for example palm oil. Hydrogenated vegetable oils also may be used in the present invention. Animal oils are also contemplated for use as glyceride esters and include, for example, fats such as tallow, lard and stearin and liquid fats, such as fish oils, fish-liver oils and other animal oils, including sperm oil, among numerous others. In addition, a number of other oils may be used, including C₁₂ to C₃₀ (or higher) fatty esters (other than the glyceride esters, which are described above) or any other acceptable oil.

The term “cosmetic ester” is used to describe any ester which is cosmetically compatible, i.e., may be safely incorporated into cosmetic products. In preferred aspects of the invention, a cosmetic ester has between 12 and 26 carbon atoms, about 14 and 20 carbon atoms within the ester compound, even more preferably about 16 and 19 carbon atoms within the ester compound, with a preferred cosmetic ester having at least one of the two chains, i.e., either the ether portion of the ester or the acyl portion of the ester being an optionally substituted (with alkyl or hydroxyl), preferably an unsubstituted branched-chain alkyl group. Preferred alkyl chains which correspond to the ether portion of the cosmetic ester include, for example, C₃-C₁₈ branched-chain alkyl groups (including alkyl groups having sany number of carbon atoms within that range), such as, for example, isopropyl, isobutyl, tert-butyl, isopentyl, neo-pentyl, branched-chain hexyl, branched-chain heptyl, branched-chain octyl, branched-chain nonyl, branched-chain decyl, branched-chain undecyl, branched-chain dodecyl, branched-chain tridecyl, branched-chain tetradecyl, branched-chain pentadecyl, branched-chain hexadecyl, branched-chain heptadecyl and branched-chain octadecyl groups linked to varying acyl groups ranging in size from C₂-C₂₂ acyl groups (including acyl groups having any number of carbon atoms within that range), accordingly. It is noted here that alternatively, the acyl group may be a branched-chain acyl group and the ether group may be an unbranched, straight chain, alkyl group. Both ether and acyl groups may comprise branched-chain alkyl groups as well.

Exemplary cosmetic esters for use in the present invention include ethyl acetate, ethyl lactate, isopropyl stearate, isopropyl palmitate, isopropyl myristate, isopropyl laurate, isopropyl oleate, isopropyl isostearate, isononyl isononanoate, isononyl isoheptanoate, isononyl isooctanoate, isododecyl isononanoate, isooctyldodecyl isononanoate, tridecyl isononanoate, decyl isononanoate, 2-ethylhexyl-2-ethylhexanoate, 2-ethylhexl isononanoate, isononyl-2-ethylhexanoate, isododecyl-2-ethylhexanoate, isodecyl-2-ethylhexanoate, decyl-2-ethylhexanoate, 2-ethylhexyl palmitate, 2-ethylhexyl myristate, 2-ethylhexyl laurate, 2-ethylhexyl decanoate, 2-ethylhexyl-2-butyloctanoate, 2-butyloctanyl-2-ethylhexanoate, capryl isopentanoate, lauryl isopentanoate, myristyl isopentanoate, palmityl isopentanoate, stearyl isopentanoate, isododecyl isononanoate, isooctyldodecyl isononanoate isododecyl neopentanoate, isooctyldodecyl neopentanoate, butyl myristate, myristyl butanoate, isostearyl isostearate, isostearyl isononanoate, isostearyl isopentanoate, isostearyl isoheptanoate, diisopropyl adipate, dioctyl adipate, diisopropyl sebacate, dioctyl sebacate, isoheptyl decanoate, isoheptyl isononanoate, isoheptyl isopentanoate, isoheptyl-2-ethylhexanoate, dicapryl maleate, di-2-ethylhexyl maleate, dicapryl fumerate, di-2-ethylhexyl fumerate, diheptyl maleate, diisononyl maleate, diheptyl fumerate and diisononyl fumerate, among numerous others.

The silicone cross-linked hydrocarbon elastomer compositions prepared above may be added to a number of additional components to produce favorable characteristics in personal care products, including skin care products, cosmetic products, antiperspirants, deodorants, perfumes, toiletries, soaps, bath oils, feminine care products, hair-care products, oral hygiene products, depilatories, including shampoos, conditioners, hair straightening products and other haircare products, color cosmetics such as lipsticks, creams, make-ups, skin creams, lotions and sunscreen products, among numerous others.

Compounds/compositions of the present invention may be used as thickening agents and emulsifiers having a number of additional characteristics including emollient and adherence characteristics for the skin and epithelial tissue such as hair, ungual tissue (nails), skin and related mucous membranes, especially given the combined attributes of emolliency (from the silicone elastomer) and skin adherence, viscosity enhancement and favorable skin interaction (generally) and wettability, enhanced solubility, UV absorbing characteristics, etc. and other attributes (which can be formulated into the polymer depending upon the inclusion of which allyl alcohol ethoxylate and/or polyurethane is chosen). By addition of an effective amount of the present compositions, emulsion formulations which may be included in personal care products, including cosmetic and toiletry products will acquire a soothing and favorable interaction which promotes skin adherence, moisturization, wettability and favorable viscosity attributes of the final personal care formulation. In addition, because the size of the silicone elastomer and polyurethane can be varied substantially, numerous characteristics may be “dialed in” to the final hydrophilic silicone elastomers in addition to the basic emulsifier characteristics and incorporated into personal care products ranging from lotions and creams to thickened formulations to be used in stick deodorants and related products can be readily formulated.

Effective amounts of the present compositions may also serve a dual function, for example, as emulsifiers exhibiting gloss-producing characteristics for lipsticks and lip balm formulations in the personal care, cosmetic and toiletry industries as a substitute(s) for castor oil normally used in such formulations, especially where the polyurethane is made from castor oil. The compounds of the present invention exhibit outstanding solubility characteristics for producing water-in-oil or oil-in-water emulsions and may form the basis for numerous and varied personal care compositions, depending upon the components of the final silicone cross-linked hydrocarbon elastomer composition.

Silicone cross-linked hydrocarbon elastomers according to the present invention exhibit one or more of a number of unexpected characteristics including providing compositions containing polyurethanes which do not exhibit a typical “sticky tactile” sensation when deposited on the skin of a subject (such as an animal, including a human) and provide a smooth, non-tacky feel which is especially advantageous for bodycare lotions and other personal care compositions used on the skin and hair of a subject. In addition, the compositions of the present invention provide “substantivity” to personal care products and can be used to accommodate functional ingredients, especially including hydrophilic functional ingredients such as polar hydrophilic materials. In certain hydrophilic silicone cross-linked hydrocarbon elastomers (which comprise allyl alcohol ethoxylate and/or hydrophilic polyurethane components) because of the hydrophilic nature of the compositions, it is easier to formulate water-in-oil emulsions, including water-in-oil in water emulsions, which results in an emulsion or final personal care composition which accommodates (on a relative scale compared to typical silicone elastomers) large amounts of water, thus reducing the cost of components and the final cost of the formulated personal care composition.

In addition, silicone cross-linked hydrocarbon elastomer compositions according to the present invention may be used advantageously as couplers (in emulsions or in compositions which are not emulsions)—for example, to couple a hydrophilic or hydrophobic component such as water and an aliphatic component (such as an oil, fatty waxes and esters) into a single formulation.

In general, silicone cross-linked hydrocarbon elastomer compositions according to the present invention are included in personal care products/formulations in effective amounts, i.e., amounts which produce an intended effect. The amount of composition generally ranges from about 0.01% to about 50% by weight or more of personal care formulations according to the present invention. Alternatively, compositions according to the present invention may be included in final personal care compositions in amounts ranging from about 0.05% to about 45% by weight, about 0.1% to about 40% by weight, about 0.25% to about 30% by weight, about 0.25% to about 20% by weight, about 0.5% to about 15% by weight, about 0.75% to about 10% by weight, about 1% to about 7.5% by weight, about 1% to about 5% by weight and about 1% to about 3% by weight of the final personal care composition.

In preferred embodiments of emulsion-based formulations (wherein the formulation comprises an oil, water and the present composition as an emulsifier, compositions according to the present invention are included in amounts ranging from about 0.1% to about 25% by weight, in addition to the oil and water and optionally, other components. Emulsions according to the present invention may be used in any number of personal care products, but find particularly useful applicability in formulations which are based upon lotions and/or skin creams.

The compositions according to the present invention may be used in numerous additional compositions. In the case of shampoos and conditioners, compositions according to the present invention are included in amounts ranging from about 0.1% to about 15% by weight of the formulation, in certain cases to instill conditioning attributes in addition to surfactant-like qualities. One can use amounts up to about 20% to 25% in shampoos and conditioners. For example, in haircare products, such as shampoos, rinses, conditioners, hair straighteners, hair colorants and permanent wave formulations, the compositions according to the present invention preferably comprise about 0.1% to about 20% by weight, more preferably about 0.25% to about 5% by weight of the final end-use hair-care composition. Other components which may be included in hair-care formulations include, for example, a solvent or diluent such as water and/or alcohol, other surfactants, emulsifiers, thickeners, coloring agents, dyes, preservatives, additional conditioning agents and humectants, among numerous others.

In the case of shave creams and gels, after-shave lotions and shave-conditioning compositions (for example, pre-electric shave formulations), the compositions according to the present invention are included in amounts ranging from about 0.25% to about 15% or more by weight, more preferably about 0.5% to about 10% by weight. Other components which may be included in these end-use compositions include, for example, water, and at least one or more of emollients, humectants and emulsifiers, thickeners and optionally, other conditioning agents, medicaments, fragrances and preservatives.

In the case of skin lotions and creams, the present compositions are included in amounts ranging from about 0.25% to about 45% by weight, more preferably, about 0.5 to about 25% by weight. Additional components which may be employed in these compositions include, for example, water, emollients and emulsifiers, surfactants, oils, and optionally, other conditioning agents, thickeners, medicaments, fragrances and preservatives.

In the case of sunscreens and skin-protective compositions, the present compositions are included in amounts ranging from about 0.25% to about 45% or more by weight, preferably about 0.5% to about 25% by weight of the final formulations. These compositions form the basis of lotions or skin creams which may be used to deliver pigments and/or sunscreen components in compositions according to the present invention. Additional components which may be employed in these compositions may include, for example, a UV absorbing composition such as para-amino benzoic acid (PABA) or a related UV absorber or a pigment such as TiO₂ and optional components including, for example, one or more of an oil, water, suspending agents, other conditioning agents and emollients, among others.

In the case of bar and liquid soaps, compositions according to the present invention are included for their surfactant and emollient-like qualities in amounts ranging from about 0.25% to about 20% by weight or more, preferably about 0.5% to about 10% by weight. Additional components which may be included in bar and liquid soaps include water and surfactants and optionally, bactericides, fragrances and colorants, among others.

Other personal care products, not specifically mentioned, generally comprise about 0.1% to about 50% by weight of a composition according to the present invention and other components of personal care products as otherwise set forth in detail herein.

In addition to the embodiments of the invention described above, in other embodiments the invention provides a cross-linked silicone elastomer gel which has a viscosity of between about 1,000 to about 100,000 centistokes (cst) and which is formed by a hydrosilylation reaction between:
(a) at least one a, co-di lower alkenyl terminated polyorganosiloxane having the formula:

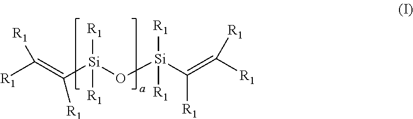

and having a molecular weight of about 20,000 to about 25,000 (preferably about 21,000 to about 24,000, more preferably about 22,000 to about 23,000, even more preferably about 22,250 to about 22,750, most preferably about 22,400 to about 22,600) with n being about 265 to about 340 (preferably about 275 to about 330, more preferably about 285 to about 320, even more preferably about 295 to about 305, still more preferably about 300) and each R₁ being independently H, or an alkyl group of 1 or 3 carbons;
(b) at least one polyorganohydrosiloxane having the formula:

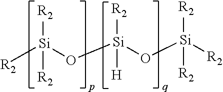

and having a molecular weight of about 3,500 to 4,000 (preferably about 3,600 to about 3,900, more preferably about 3,700 to about 3,800, still more preferably about 3,725 to about 3,775, still more preferably about 3,740 to about 3,760), where q is about 5 to about 9; p is about 40 to about 50, and each R₂ is independently an alkyl of 1-3 carbon atoms;
(c) at least one vinyl ester selected from the group consisting of cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂-C₁₃ alkyl lactate, C₁₂-C₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂-C₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxstearate or a mixture thereof; and
(d) at least one alpha-olefin of the formula (CH₂)═R³R⁴, where R³ is H or an alkyl group containing 1-40 carbon atoms, and R⁴ is an alkyl group containing 1-40 carbon atoms; and/or an alpha, omega-diene of the formula (CH₂)═CH(CH₂)ₓCH═CH₂, where x is 1-20;
   wherein the reaction (1) is conducted in the presence of a hydrosilylation catalyst and in the absence of either a solvent or a diluent (2) is induced upon addition of the at least one α,ω-di lower alkenyl terminated polyorganosiloxane, the at least one polyorganohydrosiloxane, the at least one vinyl ester and the at least one alpha-olefin to a mixer at a temperature range of between about 25° C. to about 80° C., more preferably between about 40° C. to about 50° C., even more preferably between about 40° C. to about 45° C. and (3) occurs continuously from the gel point to a point of substantial product gelation.

In still other embodiments, the invention provides a cross-linked silicone elastomer gel which has a viscosity of between about 1,000 to about 100,000 centistokes (cst) and which is formed by a hydrosilylation reaction between:
(a) at least one multi-vinyl functional hydrocarbon having the formula:

HC₃—HC═CH—CH₂—(CH₂—HC═CH—CH₂)ⱼCH₂—HC═CH—CH₃

where j is an integer from 5 to 500;
(b) at least one bis-dihydrosilane polydialkylsiloxane having the formula:

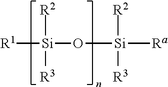

where R¹ and R^{a} are each H; each R² and R³ is independently a C₁-C₁₀ alkyl group and n is from 5 to 50,000;
(c) at least one vinyl ester selected from the group consisting of cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂-C₁₃ alkyl lactate, C₁₂-C₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂-C₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxstearate or a mixture thereof; and
(d) at least one alpha-olefin of the formula (CH₂)═R³R⁴, where R³ is H or an alkyl group containing 1-40 carbon atoms, and R⁴ is an alkyl group containing 1-40 carbon atoms; and/or an alpha, omega-diene of the formula (CH₂)═CH(CH₂)ₓCH═CH₂, where x is 1-20;
   wherein the reaction (1) is conducted in the presence of a hydrosilylation catalyst and in the absence of either a solvent or a diluent (2) is induced upon addition of the at least one multi-vinyl functional hydrocarbon, the at least one bis-dihydrosilane polydialkylsiloxane, the at least one vinyl ester and the at least one alpha-olefin to a mixer at a temperature range of between about 25° C. to about 80° C., more preferably between about 40° C. to about 50° C., even more preferably between about 40° C. to about 45° C. and (3) occurs continuously from the gel point to a point of substantial product gelation.

In still other embodiments, a, w-di lower alkenyl terminated polyorganosiloxanes and polyorganohydrosiloxanes are reacted with either at least one vinyl ester or at least one alpha-olefin. In still other embodiments, multi-vinyl functional hydrocarbons and bis-dihydrosilane polydialkylsiloxanes are reacted with either at least one vinyl ester or at least one alpha-olefin.

The following examples are intended to be illustrative of the invention concepts, and are meant to provide formulas and manufacturing methods to show some of the variations and applications that are possible.

### EXAMPLE 1

### Process Configuration

As discussed above, current hydrosilylation manufacturing processes describe reactants diluted in a solvent. This reaction yields a cross linked polymer that is soft enough to be handled using conventional processing equipment. Typically reactant levels in a diluent are low producing polymer levels of between 1-20%. The inventors have observed that a reactant blend equal to that diluted in a solvent will yield a cross linked elastomer that is dissimilar to the same reactant blend reacted without a diluent. In fact, cross linked silicone elastomers that are not internally plasticized by alpha olefins or are plasticized at ≦1% with vinyl or alkene containing reactants will yield a very hard, extremely cross linked molecule. This cross linked elastomer cannot be diluted to lower solid levels such as 5%. Diluting this elastomer will yield a gel that contains large particulates of said elastomer. The same reactant blend reacted at 20% solids level in a diluent containing ≦2% vinyl or alkene containing reactant will yield a softer less cross linked elastomer. This softer elastomer can be easily diluted to 10% and yield a very smooth gel free of large particulates of the elastomer.

The current invention utilizes a continuous process in which the reaction occurs as the reactants come into contact with each other, e.g. on the surface of a 2-24 inch mixing trough. Once the reaction is complete the next step of the process, milling to a smaller particle size or milling and dilution of the concentrate can occur. This is done without the waiting period described in current processes. Analysis confirms that there is no need to allow the cross-linked silicone elastomer to sit without mixing so that the reaction comes to completion. Iodine values in conjunction with NMR analysis confirm total consumption of all reactants containing vinyl or alkenyl functional groups. If all reactants are consumed, a resting period of the gel is unnecessary. If one of the reactants is totally consumed, then there can be no continuation of cross linking and again no rest period is necessary. Common practice describes a mixing vessel. The current invention will describe a reaction that occurs as the reactants come into contact with each other in a small reaction environment/surface. The reaction will occur on the surface of the reaction vessel and not in a large volume environment.

The current invention describes a mixing of the alkenyl esters and or vinyl containing compounds and catalyst in one vessel. This blend can be called “Blend A”. Blend A can be 100% mixture of reactants and catalyst. This blend can also contain a solvent or diluent. Solvent or diluent for the purpose of this description can be further defined by its non-reactive state. The preferred embodiment contains no solvent or diluent but not limited to a non-solvent reaction. A second vessel containing silanic hydrogen is required. This second reactant blend can be called “Blend B”. The current process can react and yield a product at temperature ranges of 25-80° C. Preferred temperature range utilizing the continuous manufacturing process is 40-50° C. The reactants contained in “Blend A” and “Blend B” are mixing while maintaining a 40-45° C. temperature. In one embodiment the Blends, A and B do can be mixed and reacted at room temperature ≈25° C. Hydrosilylation typically requires elevated temperatures so that the reaction can proceed quickly. The current invention utilizes a continuous process reactor that can heat the reactants as they mix on the surface of the reactor vessel.

The two blends are dosed into a “Continuous Processor” such as the ones manufactured by Readco Kurimoto, LLC (York, Pa.). This “Continuous Processor” can be described as a mixer/extruder. The reaction vessel can be described as a vertically aligned trough. This trough can be a 2 inch diameter up to a 24 inch diameter. The trough contains two screw shafts that can be configured with mixing paddles. These paddles can be configured to mix or extrude. The trough also has the flexibility to allow for the addition of reactants, solvents, and other ingredients in a liquid or solid state. The trough can be segmented to have a half that can be heated while the second half of the trough can be cooled.

Dosing of Blend A+B are performed in such a manner as to achieve complete reaction of the reactants. This reaction produces a rubber gel that can be continuously mixed and extruded out the other end of the continuous process mixer. Prior art and the utilization of common mixing vessels will not allow for the continuation of mixing or agitation. Current practice typically describes a rest period wherein the product of this reaction is allowed to sit with no mixing to complete the reaction. The reaction environment is small in the invention described herein. Point of mixing is the point of reaction. Reaction time occurs at the point of mixing on the surface of the reactor vessel or reactor “trough”.

### EXAMPLE 2

### Cross-Linked Silicone Elastomer Gels

The manufacturing/reaction process defined in this invention has led to surprising results. Using the process described in this invention gels were made using higher viscosity Dimethicone than were thought possible utilizing the conventional manufacturing method. The new process can make gels diluted in Dimethicone that are 1,000-100,000 cst. Current conventional process could not make a gel higher than 300 cst. The viscosity of the diluent had a limiting effect on the cross linking reaction.

As the cross linking reaction proceeds, viscosity will increase. The viscosity of the diluent allows the crosslinking to occur throughout the reaction vessel. If the viscosity is high enough (>300 cst using the conventional process in a large reaction vessel) the cross linking will not spread throughout the vessel. The reaction using a continuous processor is not hindered by the viscosity of the diluent or even the viscosity of the reactants. The viscosity of the product as the reactants react will not be a limiting factor as well.

The reaction environment in the continuous processor is very small. To make 1,000 lbs. of a cross linked silicone elastomer in the conventional manner. We would need a vessel that can hold the 1,000 lbs. The reactants need to be mixed or agitated at a certain temperature. If the reactants were catalyzed and reacted without a diluent the reaction would yield a product that was very viscous, sometimes tacky, and typically a hard rubber. The resulting rubber has to be removed from the vessel and without a diluent this could be difficult to impossible. This rubber would then require milling and dilution to get a homogenous material.

The continuous processor has the ability to produce up to but not limited to 1,000 lbs. of material with solvent or without solvent. The continuous process splits the reactant blend into 2 parts. One part contains vinyl silicones, alkenyl esters and or alpha olefins and catalyst. The second part contains silanic hydrogen. These are dosed into the continuous processor at a certain rate. The reaction and therefore cross linking reaction occurs as the two reactant blends come into contact with each other. This reaction is occurring at but not limited to 1-5 lbs. a second. The throughput of material noted utilized a 5 inch trough/reactor manufactured by Readco Kurimoto. The larger diameter reactors would increase this throughput.

As the crosslinking is reaching a non-Newtonian viscosity the continuous processor acts as an extruder and continuous to knead or mix the silicone elastomer until it is extruded into a holding vessel. In the conventional process all mixing or agitation is stopped and the reaction proceeds for several hours in the vessel without mixing or agitation. This is common practice for achieving complete cross linking.

Continuous process allows for mixing after the gelation point of the reaction. Once the gel is extruded out of the continuous processor the gel is ready for further dilutions. This can be accomplished with a homogenizer such as those made by Silverson. Preferably an inline homogenizer which ensures an efficient and homogenous particle distribution of the silicone cross linked gel.

The addition of alkenyl esters or vinyl compounds can be used to not only affect compatibility parameters but the physical properties of the gel itself. The following is a listing of possible reactant mixtures.

Hydrosilylation reaction combinations:
1. Bis-vinyl silicones+Silicon-hydride containing polysiloxanes (each reactant is available at different molecular weights)
2. Combination #1 mixed molecular weights, ex. a bis-vinyls at different molecular weights+Silicon-hydride containing polysiloxanes where a>1
3. Combination #1 mixed molecular weights, ex. Bis-vinyl+b Silicon-hydride containing polysiloxanes at different molecular weights where b>1
4. Combination #1 mixed molecular weights, ex. a Bis-vinyl at different molecular weights+b Silicon-hydride containing polysiloxanes at different molecular weights. Where the ratios of a and b are a>1 and b>1
5. Bis-vinyl silicones+Silicon-hydride containing polysiloxanes+αolefins (each reactant is available at different molecular weights)
6. Combination #5 mixed molecular weights, same as in Combinations 2-4 including mixed molecular weights. Ratios of the α olefins can be >1
7. Bis-vinyl silicones+Silicon-hydride containing polysiloxanes+olefins (each reactant is available at different molecular weights and isomers of olefins).
8. Combination #7, same as in Combinations 2-4 including mixed molecular weights and isomers of olefins. Ratios of the olefins can be >1
9. Bis-vinyl silicones+Silicon-hydride containing polysiloxanes+esters containing alkenes (each reactant is available at different molecular weights and isomers of esters containing alkenes)
10. Combination #9, same as in Combinations 2-4 including mixed molecular weights and isomers of esters containing alkenes. Ratios of the esters containing alkenes can be >1
11. Bis-vinyl silicones+Silicon-hydride containing polysiloxanes+polyenes (each reactant is available at different molecular weights and isomers of polyenes)
12. Combination #11, same as in Combinations 2-4 including mixed molecular weights and isomers of the polyenes. Ratios of the polyenes containing alkenes can be >1
13. Bis-vinyl silicones+Silicon-hydride containing polysiloxanes+Vinyl silicones (each reactant is available at different molecular weights)
14. Combination #13 mixed molecular weights, same as in Combinations 2-4 including mixed molecular weights. Ratios of the vinyl silicones can be >1
15. Bis-vinyl silicones+Silicon-hydride containing polysiloxanes+polyvinyl silicones (each reactant is available at different molecular weights)
16. Combination #15 mixed molecular weights, same as in Combinations 2-4 including mixed molecular weights. Ratios of the polyvinyl silicones can be >1
17. Bis-vinyl silicones+Silicon-hydride containing polysiloxanes+allyl alcohols (each reactant is available at different molecular weights)
18. Combination #17 mixed molecular weights, same as in Combinations 2-4 including mixed molecular weights. Ratios of the polyvinyl allyl alcohols can be >1
19. Bis-vinyl silicones+Silicon-hydride containing polysiloxanes+vinyl alcohols (each reactant is available at different molecular weights)
20. Combination #19 mixed molecular weights, same as in Combinations 2-4 including mixed molecular weights. Ratios of the vinyl alcohols can be >1
   Silicon-hydride containing polysiloxanes can be used that contain pendant, terminal, and hybrids containing both terminal and pendant functionality. Terminal functionality is used as chain extenders. Pendant functionality is used as cross linkers. Hybrids which contain terminal and pendant functionality increase cross linking density.
   The following table discloses Hydrosilylation reactions.
A) Experiment #BB 1-34B describes a cross linked silicone elastomer that contains 3.5% α-olefin grafted onto the silicone elastomers backbone. This elastomer has a clear appearance. The elastomer is non-Newtonian and very tacky to the touch. This elastomer can be diluted to 50% in Isododecane and behave as a Newtonian fluid. The fluid is clear and has a viscosity of 1,000 cps. This elastomer diluted in Isododecane can be applied to a substrate leaving a shiny adhesive film on the skin, hair, nail, woven and no-woven fabrics, leather, and wood, plastic, stone or other substrates. It has a great adhesion, water proofing as well as conditioning properties.
B) Experiment #MB1-225E describes a cross linked silicone elastomer that contains 7.25% Mango Butter Dimer Dilinoleyl Esters/Dimer Dilinoleate Copolymer. This elastomer is opaque and white in appearance. It is a hard non tacky rubber. This elastomer when diluted to 12% in Isododecane yields a thick flow able paste with a viscosity of 30,000 cps. The white opaque appearance of this elastomer is due to an incompatibility of the silicon hydride, bis-vinyl Polydimethylsiloxane, and the Mango Butter Dimer Dilinoleyl Esters/Dimer Dilinoleate Copolymer. This elastomer diluted to 12% in Isododecane is clear.

In the first experiment BB1-34B, we are able to increase the level of C18 α-olefin to 3.5%. The addition of α-olefins >1.0% and reacted without a diluent yields gels that are soft and tacky. Manufacturing large quantities of these elastomers using conventional processes as described in the current art is very difficult to impossible. Elastomer experiment BB1-34B produces a clear, soft, and very tacky gel. The elastomers sticks to stainless steel and would be very difficult to transfer from one vessel to another. A continuous process as described in this invention would be able to produce and move this gel form one vessel to another.

In the second experiment MB1-225E we are grafting an alkene containing ester, Mango Butter Dimer Dilinoleyl Esters/Dimer Dilinoleate Copolymer. This reaction was made at 100% reactant levels with no diluent. The white opaque hard rubber denotes a slight incompatibility within this molecule. This reaction would not be able to be manufactured under the typical conditions described in the art. A continuous process as described would be able to react, produce and move this hard rubber. The continuous process described in this invention would mill this hard rubber after the reaction takes place and yield a smaller particle size rubber ready for dilution and further reduction of the particle size.

Another point of difference between BB1-34B, a cross linked silicone elastomer containing an, α-olefin and MB 1-225E a cross linked silicone elastomer containing an alkene containing ester is the location of the alkene functionality. A, α-olefin contains an alkene at the end of the molecule whereas the ester's alkene functionality is located somewhere along the middle of the molecule. The α-olefin once grafted onto the silicon hydride is a single linear pendant group. The ester will react along the middle of the molecule and will produce two linear pendant groups attached to the silicon hydride. The ester grafted onto the silicon hydride produce a white opaque rubber gel. MB1-225E diluted to 12% in Isododecane makes a clear gel. The dual alkyl pendant group made by reacting an ester to silicon hydride is not as compatible as the single alkyl group produced by using a α-olefin.

Experiment MB1-227A is a cross-linked silicone elastomer that contains 4% C18 α-olefin and 3.25% Dimer Diliniloeate grafted onto the elastomer. This reaction produces a clear soft tacky rubber. This elastomer is a clear viscous, 3,000 cps, liquid.

| ID Gel | | | | | | | |
|---|---|---|---|---|---|---|---|
| (Experiment | | | Internal | Iodine | NMR | | |
| Number) | V2K | SiH | Plastisizer(s) | Value | results | Comments | Application |
| BB1-34A | 69.5 | 27 | AOC-12: 3.5 | 6.01 | | | |
| BB1-34B | 69.5 | 27 | AOC-18: 3.5 | 7.29 | | extremely | lipstick-used |
| | | | | | | sticky, | 25% in IDD. |
| | | | | | | soluble in | Very soft feel |
| | | | | | | IDD and D99 | |
| BB1-34C | 72 | 27 | AOC-12: 1 | 9.13 | | | |
| BB1-34D | 72 | 27 | AOC-18: 1 | 9.98 | | | |
| BB1-36A | V- | 27 | AOC-12: 3.5 | 6.17 | | similar to | |
| | 1,000: | | | | | BB!-34A | |
| | 69.5 | | | | | | |
| MB1-220A | 75 | 24.984 | AOC-12: 10 | 3.81 | | | |
| MB1-223A | 65.75 | 27 | AOC-12: 7.25 | 1.51 | | several | |
| | | | | | | lipglosses | |
| | | | | | | were made | |
| | | | | | | using 50% in | |
| | | | | | | IDD, good | |
| | | | | | | application | |
| | | | | | | and stay | |
| MB1-224A | 65.75 | 27 | AOC-18: 7.26 | 1.81 | | | Slightly |
| | | | | | | | chunky/unstable |
| MB1-224C | 65.75 | 27 | AOC-20-24: | 4.12 | | | looks unstable, |
| | | | 7.25 | | | | slightly chunky |
| MB1-224D | 65.75 | 27 | AOC-26-28: | 1.41 | | | |
| | | | 7.25 | | | | |
| MB1-225A | 65.75 | 27 | AOC-12: 3; | 3.08 | | | sheer, stable, |
| | | | AOC-18: 2.25; | | | | good |
| | | | AOC-20-24: 2 | | | | application |
| MB1-225B | 65.75 | 27 | DID: 7.25 | 1.07 | | | |
| MB1-225C | 65.75 | 27 | GRC: 7.25 | 1.62 | | | |
| MB1-225E | 65.75 | 27 | MBDD: 7.25 | 7.62* | | | |
| MB1-226A | 65.75 | 27 | DID: 3.25; | 5.7 | | | |
| | | | AOC-12: 4 | | | | |
| MB1-226B | 65.75 | 27 | GRC: 3.25; | 7.62* | | | |
| | | | AOC-12: 4 | | | | |
| MB1-226C | 65.75 | 27 | Castor Oil: 7.25 | 7.22* | | | |
| MB1-226D | 65.75 | 27 | Polyderm PPI- | 6.1* | | | |
| | | | CO: 7.25 | | | | |
| MB1-226E | 65.75 | 27 | Sesame Oil: | 17.21* | | | |
| | | | 7.25 | | | | |
| MB1-227A | 65.75 | 27 | AOC-18: 4; | 7.5 | | | |
| | | | DID: 3.25 | | | | |
| MB1-227B | 65.75 | 27 | AOC-12: 2; | 8.91; 4.99 | | | |
| | | | DID: 5.25 | diluted at | | | |
| | | | | 50% | | | |
| MB1-228A | 67.5 | 29 | AOC-12: 3.5 | 4.99 | | | |
| PEG 2- | | | | 27.44 | | | |
| Soyamine | | | | | | | |

*gel did not dissolve during testing

What is claimed:

1. A cross-linked silicone elastomer gel product formed by a hydrosilylation reaction between:
(a) at least one unsaturated polyorganosiloxane;
(b) at least one polyorganohydrosiloxane; and
(c) optionally, at least one vinyl ester and/or alpha olefin;
wherein the reaction (1) is conducted in the presence of a hydrosilylation catalyst and, optionally, a solvent or diluent (2) is induced upon addition of the at least one unsaturated polyorganosiloxane and the at least one polyorganohydrosiloxane to a mixer at a temperature range of between about 25° C. to about 80° C., more preferably between about 40° C. to about 50° C., even more preferably between about 40° C. to about 45° C. and (3) occurs continuously from the gel point to a point of substantial product gelation.

2. The cross-linked silicone elastomer gel of claim 1, wherein the silicone elastomer gel has a viscosity of between about 1,000 to about 3,000,000 centistokes (cst).

3. The cross-linked silicone elastomer gel of claim 1, wherein the hydrosilylation reaction is conducted in the presence of a solvent or diluent.

4. The cross-linked silicone elastomer gel of claim 1, wherein the silicone elastomer gel is a Newtonian or non-Newtonian gel.

5. The cross-linked silicone elastomer gel of claim 1, wherein:
(a) the at least one unsaturated polyorganosiloxane is a bis-divinyl polydimethylsiloxane or a bis-divinyl polymethylphenylsiloxane; and
(b) the at least one polyorganohydrosiloxane is a polydimethylsiloxane having multiple pendant methylhydrosilane or phenylhydrosilane groups.

6. The cross-linked silicone elastomer gel of claim 5, wherein the hydrosilylation reaction is conducted in the presence of a solvent or diluent.

7. The cross-linked silicone elastomer gel of claim 5, wherein the silicone elastomer gel is a Newtonian or non-Newtonian gel.

8. The cross-linked silicone elastomer gel of claim 1, wherein:
(a) the at least one unsaturated polyorganosiloxane is an α,ω-di lower alkenyl terminated polyorganosiloxane having the formula: 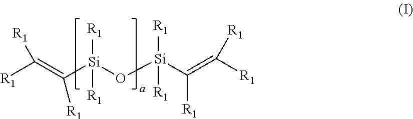
and having a molecular weight of about 20,000 to about 25,000 (preferably about 21,000 to about 24,000, more preferably about 22,000 to about 23,000, even more preferably about 22,250 to about 22,750, most preferably about 22,400 to about 22,600) with n being about 265 to about 340 (preferably about 275 to about 330, more preferably about 285 to about 320, even more preferably about 295 to about 305, still more preferably about 300) and each R₁ being independently H, or an alkyl group of 1 or 3 carbons; and
(b) the at least one polyorganohydrosiloxane has the formula: 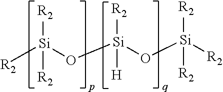
and a molecular weight of about 3,500 to 4,000 (preferably about 3,600 to about 3,900, more preferably about 3,700 to about 3,800, still more preferably about 3,725 to about 3,775, still more preferably about 3,740 to about 3,760), where q is about 5 to about 9; p is about 40 to about 50, and each R₂ is independently an alkyl of 1-3 carbon atoms.

9. The cross-linked silicone elastomer gel of claim 8, wherein the hydrosilylation reaction is conducted in the presence of a solvent or diluent.

10. The cross-linked silicone elastomer gel of claim 8, wherein the silicone elastomer gel is a Newtonian or non-Newtonian gel.

11. The cross-linked silicone elastomer gel of claim 1, wherein:
(a) the at least one unsaturated polyorganosiloxane is a bis-divinyl polydimethylsiloxane; and
(b) the at least one polyorganohydrosiloxane is a polydimethylsiloxane having multiple pendant methylhydrosilane groups.

12. A cross-linked silicone elastomer gel formed by a hydrosilylation reaction between:
(a) at least one alpha-olefin of the formula (CH₂)═R³R⁴, where R³ is H or an alkyl group containing 1-40 carbon atoms, and R⁴ is an alkyl group containing 1-40 carbon atoms; and/or an alpha, omega-diene of the formula (CH₂)═CH(CH₂)ₓCH═CH₂, where x is 1-20; and
(b) at least one polydimethylsiloxane having multiple pendant methylhydrosilane groups;
wherein the reaction (1) is conducted in the presence of a hydrosilylation catalyst and, optionally, a solvent or diluent (2) is induced upon addition of the at least one alpha-olefin and the at least one polydimethylsiloxane to a mixer at a temperature range of between about 25° C. to about 80° C., more preferably between about 40° C. to about 50° C., even more preferably between about 40° C. to about 45° C. and (3) occurs continuously from the gel point to a point of substantial product gelation.

13. The cross-linked silicone elastomer gel of claim 12, wherein the silicone elastomer gel has a viscosity of between about 1,000 to about 3,000,000 centistokes (cst).

14. The cross-linked silicone elastomer gel of claim 12, wherein the hydrosilylation reaction is conducted in the presence of a solvent or diluent.

15. The cross-linked silicone elastomer gel of claim 12, wherein the silicone elastomer gel is a Newtonian or non-Newtonian gel.

16. A cross-linked silicone elastomer gel formed by a hydrosilylation reaction between:
(a) at least one multi-vinyl functional hydrocarbon;
(b) at least one bis-dihydrosilane polydialkylsiloxane; and
(c) optionally, an allyl alcohol ethoxylate;
wherein the reaction (1) is conducted in the presence of a hydrosilylation catalyst and, optionally, a solvent or diluent (2) is induced upon addition of the at least one multi-vinyl functional hydrocarbon, the at least one bis-dihydrosilane polydialkylsiloxane and, optionally, the at least one ally! alcohol ethoxylate to a mixer at a temperature range of between about 25° C. to about 80° C., more preferably between about 40° C. to about 50° C., even more preferably between about 40° C. to about 45° C. and (3) occurs continuously from the gel point to a point of substantial product gelation.

17. The cross-linked silicone elastomer gel of claim 16, wherein the silicone elastomer gel has a viscosity of between about 1,000 to about 3,000,000 centistokes (cst).

18. (canceled)

19. (canceled)

20. The cross-linked silicone elastomer gel of a claim 16, wherein the multi-vinyl functional hydrocarbon has the formula:
HC₃—HC═CH—CH₂—(CH₂—HC═CH—CH₂)CH₂—HC═CH—CH₃
where j is an integer from 5 to 500.

21. (canceled)

22. The cross-linked silicone elastomer gel of claim 16, wherein the bis-dihydrosilane polydialkylsiloxane has the formula: 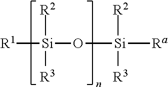
where R¹ and R^{a} are each H; each R² and R³ is independently a C₁-C₁₀ alkyl group; and
n is from 5 to 50,000.

23. The cross-linked silicone elastomer gel claim 16, wherein the bis-dihydrosilane polydialkylsiloxane comprises about 0.01% to about 7.5% b_{y} weight of allyl alcohol ethoxylate units.

24. The cross-linked silicone elastomer gel of claim 16, wherein each of said allyl alcohol ethoxylate units comprises about 5 to about 100 ethylene glycol units.

25. The cross-linked silicone elastomer gel of claim 16, wherein the bis-dihydrosilane polydialkylsiloxane comprises about 0.01% to about 7.5% by weight of a polyurethane.

26. A cross-linked silicone elastomer gel which has a viscosity of between more than 100,000 centistokes (cst) to 4,000,000 centistokes and which is formed by a hydrosilylation reaction between:
(a) at least one α,ω-di lower alkenyl terminated polyorganosiloxane having the formula: 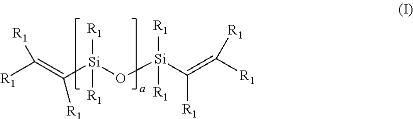
and having a molecular weight of about 20,000 to about 25,000 (preferably about 21,000 to about 24,000, more preferably about 22,000 to about 23,000, even more preferably about 22,250 to about 22,750, most preferably about 22,400 to about 22,600) with n being about 265 to about 340 (preferably about 275 to about 330, more preferably about 285 to about 320, even more preferably about 295 to about 305, still more preferably about 300) and each R₁ being independently H, or an alkyl group of 1 or 3 carbons;
(b) at least one polyorganohydrosiloxane having the formula: 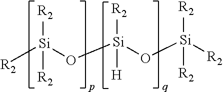
and having a molecular weight of about 3,500 to 4,000 (preferably about 3,600 to about 3,900, more preferably about 3,700 to about 3,800, still more preferably about 3,725 to about 3,775, still more preferably about 3,740 to about 3,760), where q is about 5 to about 9; p is about 40 to about 50, and each R₂ is independently an alkyl of 1-3 carbon atoms;
(c) at least one vinyl ester selected from the group consisting of cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂-C₁₃ alkyl lactate, C₁₂-C₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂-C₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxstearate or a mixture thereof; and
(d) at least one alpha-olefin of the formula (CH₂)═R³R⁴, where R³ is H or an alkyl group containing 1-40 carbon atoms, and R⁴ is an alkyl group containing 1-40 carbon atoms; and/or an alpha, omega-diene of the formula (CH₂)═CH(CH₂)ₓCH═CH₂, where x is 1-20;
wherein the reaction (1) is conducted in the presence of a hydrosilylation catalyst and in the absence of either a solvent or a diluent (2) is induced upon addition of the at least one α,ω-di lower alkenyl terminated polyorganosiloxane, the at least one polyorganohydrosiloxane, the at least one vinyl ester and the at least one alpha-olefin to a mixer at a temperature range of between about 25° C. to about 80° C., more preferably between about 40° C. to about 50° C., even more preferably between about 40° C. to about 45° C. and (3) occurs continuously from the gel point to a point of substantial product gelation.

27-29. (canceled)

30-33. (canceled)

34. A process for making a cross-linked silicone elastomer gel product, the process comprising a hydrosilylation reaction in a mixer between:
(a) at least one unsaturated polyorganosiloxane;
(b) at least one polyorganohydrosiloxane; and
(c) optionally, at least one vinyl ester and/or alpha olefin;
wherein the hydrosilylation reaction (1) is conducted in the presence of a hydrosilylation catalyst and, optionally, a solvent or diluent (2) is induced upon addition of the at least one unsaturated polyorganosiloxane and the at least one polyorganohydrosiloxane to the mixer at a temperature range of between about 25° C. to about 80° C., more preferably between about 40° C. to about 50° C., even more preferably between about 40° C. to about 45° C. (3) occurs continuously from the gel point to a point of substantial product gelation, and wherein (4) the at least one vinyl ester and/or alpha olefin, if present, is blended with the at least one unsaturated polyorganosiloxane prior to addition to the mixer.

35-40. (canceled)

41. The process of claim 34 or 35, wherein:
(a) the at least one unsaturated polyorganosiloxane is an α,ω-di lower alkenyl terminated polyorganosiloxane having the formula: 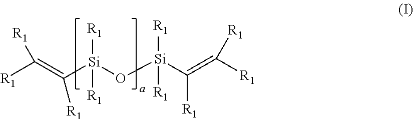
and having a molecular weight of about 20,000 to about 25,000 (preferably about 21,000 to about 24,000, more preferably about 22,000 to about 23,000, even more preferably about 22,250 to about 22,750, most preferably about 22,400 to about 22,600) with n being about 265 to about 340 (preferably about 275 to about 330, more preferably about 285 to about 320, even more preferably about 295 to about 305, still more preferably about 300) and each R₁ being independently H, or an alkyl group of 1 or 3 carbons; and
(b) the at least one polyorganohydrosiloxane has the formula: 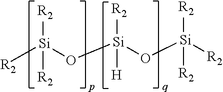
and a molecular weight of about 3,500 to 4,000 (preferably about 3,600 to about 3,900, more preferably about 3,700 to about 3,800, still more preferably about 3,725 to about 3,775, still more preferably about 3,740 to about 3,760), where q is about 5 to about 9; p is about 40 to about 50, and each R₂ is independently an alkyl of 1-3 carbon atoms.

42-44. (canceled)

45. A process for making a cross-linked silicone elastomer gel product, the process comprising a hydrosilylation reaction in a mixer between:
(a) at least one alpha-olefin of the formula (CH₂)═R³R⁴, where R³ is H or an alkyl group containing 1-40 carbon atoms, and R⁴ is an alkyl group containing 1-40 carbon atoms; and/or an alpha, omega-diene of the formula (CH₂)═CH(CH₂)ₓCH═CH₂, where x is 1-20; and
(b) at least one polydimethylsiloxane having multiple pendant methylhydrosilane groups;
wherein the reaction (1) is conducted in the presence of a hydrosilylation catalyst and, optionally, a solvent or diluent (2) is induced upon addition of the at least one alpha-olefin and the at least one polydimethylsiloxane to the mixer at a temperature range of between about 25° C. to about 80° C., more preferably between about 40° C. to about 50° C., even more preferably between about 40° C. to about 45° C. and (3) occurs continuously from the gel point to a point of substantial product gelation.

46-79. (canceled)
